(19) United States
(12) Patent Application Publication
(10) Pub. No.: US 2014/0306070 A1
(43) Pub. Date: Oct. 16, 2014

(54) IV Line Organizer

(71) Applicant: LMH Products, LLC, Edon, OH (US)

(72) Inventors: Timothy Hartsock, Edon, OH (US); Douglas Michael, Edon, OH (US)

(73) Assignee: LMH Products, LLC, Edon, OH (US)

(21) Appl. No.: 14/248,978

(22) Filed: Apr. 9, 2014

Related U.S. Application Data
(60) Provisional application No. 61/853,828, filed on Apr. 11, 2013.

(51) Int. Cl. A61M 5/14

(52) U.S. Cl. CPC A61M 5/1418
USPC 248/68.1

(57) ABSTRACT

Versions of the present invention comprise an apparatus for organizing and retaining IV lines in a device attachable to a bed rail, IV post, wheelchair arm rail, or other apparatus.

This Application claims priority to provisional U.S. Application No. 61/853,828, IV Line Organizer by Timothy Hartsock, filed on Apr. 11, 2013.

### STATEMENT REGARDING FEDERALLY SPONSORED RESEARCH OR DEVELOPMENT

Not applicable

### BACKGROUND

Intravenous lines, known as “IV” lines, are an important and commonly-used apparatus in the provision of medical care. IV lines are employed to infuse liquids directly into a patient's bloodstream over an extended period of time. IV lines can be used as part of a broad array of treatments, including, by way of example, administering saline solution to provide basic hydration, administering medication at stated time intervals or over a long period of time or administering painkillers on a schedule or on demand.

As will be appreciated by one skilled in the art, IV lines are typically connected to a catheter, which can be inserted into a blood vessel, most commonly a peripheral vein, by piercing. In many cases, an IV line is attached through a catheter to a patient's hand or forearm. IV lines are commonly attached at their opposite end to a fluid reservoir. Fluid from the reservoir is placed under pressure from one or more of gravity, pumps, or other means known to those skilled in the art. Fluid is forced from the fluid reservoir through the IV line to the patient either on a continuous basis, on a predetermined schedule, or upon the occurrence of pre-defined conditions. In many cases, patients will be attached to multiple IV lines, which are in turn attached to multiple fluid reservoirs.

IV lines are commonly of substantial length to allow the patient to have some freedom of movement, and to allow the fluid reservoirs to be located at a location remote from the patient so as to reduce their interference with the provision of other medical services or the movement of service providers. As a consequence, in a hospital setting, IV lines commonly run from a patient to a location outside of the patient's bed. One major drawback of IV lines is that they in many cases hang, run, or are otherwise located in places where they can snag or become entangled with the patient, visitors, service providers, or other equipment, including the patient's bed, or where they can pose an obstacle or trip hazard for individuals moving through the room. Another drawback to IV lines is that they can be inadvertently disconnected from the patient's body or from a fluid reservoir if they are subjected to significant external force, such as being tripped over, stepped on, jostled, or entangled. Such disconnection can be acutely painful, as well as deleterious to the patient's health through the unintended cessation of whatever fluid the IV line had been providing. Another drawback to IV lines is that they can become twisted, pinched, kinked, or otherwise rendered inoperable without being disconnected. These drawbacks, and the risk of tripping, entanglement, and kinking, become significantly pronounced when a patient is attached to multiple IV lines.

The present invention addresses these drawbacks by providing an apparatus adapted to assist with the organization, separation, and security of one or more IV lines. The apparatus of the present invention is removably attachable to commonly-utilized hospital equipment, such as an IV pole, a wheelchair handle, or, preferably, a patient's bed. The present invention further keeps multiple IV lines separated but clearly identified, reduces the risks of tripping, entanglement, and kinking, and allows IV lines to he added or removed as desired.

### SUMMARY

The apparatus of the present invention comprises a base plate, said base plate further comprising a plurality of line hooks, and still further comprising one or more connectors attached to the base plate configured for removable attachment to other structures, including but not limited to an IV pole, wheelchair arm, or hospital bed. Each line hook is adapted for removable attachment to a one or more IV lines. The connectors arc configured to removably attach the apparatus to a hospital bed, and preferably to the bed rail.

In a preferred embodiment, the plurality of line hooks are configured in series and are integrally molded into the base plate. The one or more connectors are additionally integrally molded into the baseplate In this preferred embodiment, the connectors are configured for removable attachment to a bed rail, and the apparatus is made from plastic of sufficient rigidity to allow a deformable snap on connection between the one or more connectors and a bed rail, and is further of sufficient rigidity to allow a deformable snap-in friction fit between each line hook and one or more IV lines when said one or more IV lines are pressed into the line hook.

### BRIEF DESCRIPTION OF THE DRAWINGS

The aforementioned and other features and objects of the present invention and the manner of attaining them will become more apparent and the invention itself will be best understood by reference to the following descriptions of a preferred embodiment and other embodiments taken in conjunction with the accompanying drawings, wherein:

FIG. 1 shows a perspective view of one embodiment of the present invention attached to a bed rail;

FIG. 2 shows a front cross-sectional view of one embodiment of the present invention;

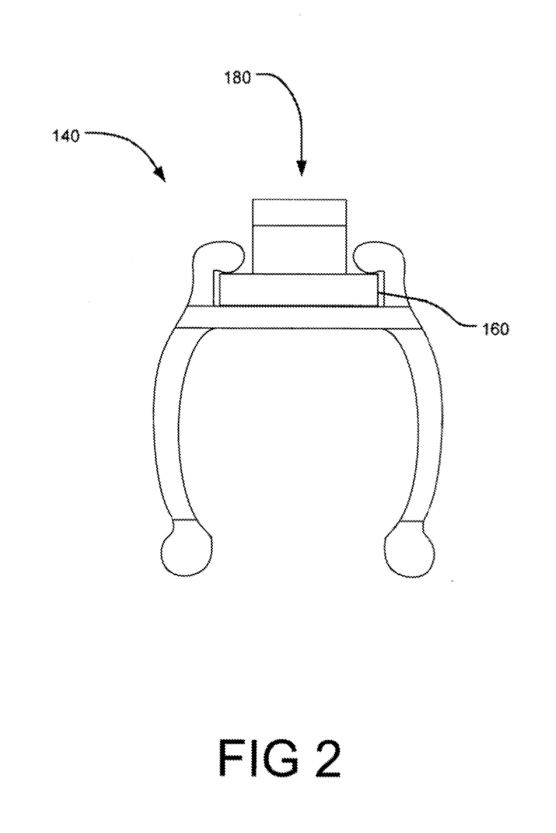

FIG. 3 shows a side view of one embodiment of the present invention; and

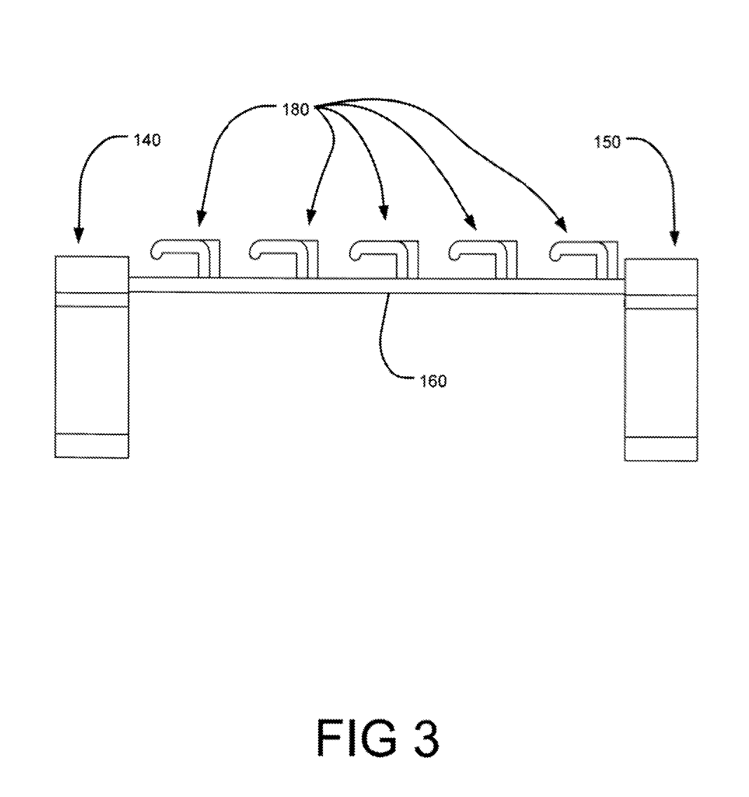

FIG. 4 shows a side view of one line hook in one embodiment of the present invention holding two IV lines.

### DETAILED DESCRIPTION

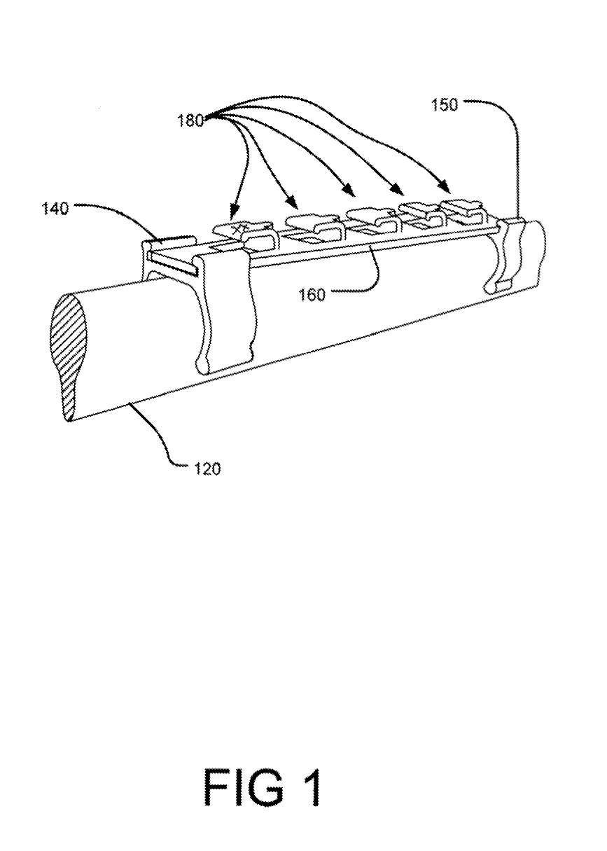

Referring now to the drawings. FIG. 1 illustrates an embodiment of the IV line organizer removably attachable to and attached to a bed rail (120). The IV line organizer comprises a base plate (160), one or more connectors (140, 150) attached to said base plate (160) and adapted for connection to a bed rail (120), and plurality of line hooks (180) connected said base plate (160).

A base plate (160) according to the teachings hereof is a plate adapted to present a plurality of fine hooks (180), and further configured to comprise one or more connectors (140, 150) such that when said base plate (160) is connected to a bed rail (120) by said one or more connectors (140, 150), said line hooks (180) are placed into an orientation suitable for use in holding and organizing IV lines. A base plate (160) can be of any size and made of any material suitable for use in a medical services environment, and particularly a hospital environment. Preferably, said base plate (160) is made of thermoform or thermoset plastic of sufficient structural strength and rigidity to prevent said base plate (160) from bending, bowing, or cracking when the apparatus is in use.

In the embodiment shown in FIG. 1, said connectors comprise a first clamp (140) and a second clamp (150) made of flexible material such as plastic, adapted for attachment to said bed rail (120) through a friction fit created when said first clamp and said second clamp are pressed downward onto the bed rail (120) until the arms of each clamp deform to accept the rail and then snap back into position to hold the rail in place. As will be understood by one skilled in the art, however, virtually any physical connector known to the art appropriate for the removable connection of one physical object to another may be used within the scope and spirit of this invention. Others suitable connectors include, without limitation, threaded connectors, threaded clamps, spring-biased clamps, hook-and-loop connectors, hooks, C-clamps, ties, and magnetic connectors. As will be further understood by one skilled in the art, while the present invention is configured to removably attach to a bed rail by way of clamps in the preferred embodiment, it may in other embodiments removably attach to other structures by way of other connectors. For example, in another embodiment, the connectors comprise one or more ring clamps and the apparatus is configured for removable attachment to an IV pole. For further example, in yet another embodiment, the connectors comprise one or more threadably adjustable C-clamps and the apparatus is configured for removable attachment to a wheelchair arm rail. Other connector types and other configurations for attachment to other furniture or structures commonly found in hospitals and other care settings within the scope and spirit of this invention will be apparent to one skilled in the art.

In some embodiments, the base plate (160) attaches to the one or more connectors through a mechanical connection means, as would be appreciated by one skilled in the art. Appropriate connection means would include, but not be limited to, friction fit, threaded connection, pins, rivets, hook-and-loop connection, zipper connection, snaps, books, clamps, ties, or buttons. In the embodiment shown in FIG. 1, the base plate (160) attaches to the one or more connectors through a friction fit. In other embodiments, the one or more connectors may be permanently attached to said base plate (160). In still other embodiments, the base plate (160) may comprise said one or more connectors or said one or more connectors may be integrally molded or formed into said base plate (160).

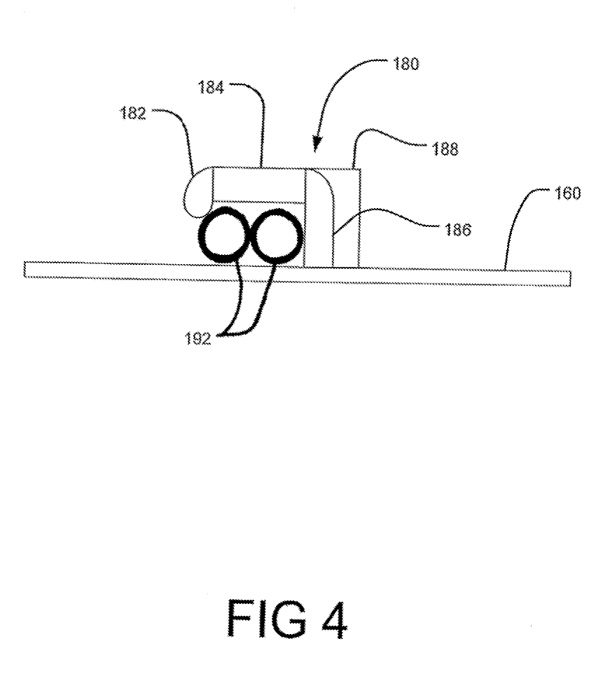

The base plate (160) further comprises a plurality of line hooks (180). Each line hook (180) is adapted to hold one or more IV lines. In a preferred embodiment as shown in FIG. 4, each line hook (180) is adapted to hold two IV lines. A line hook (180) according to the teachings of this invention is a structure adapted to removably hold an IV line in place. As shown in FIG. 4, a line hook (180) comprises an upright member (186), a top member (184), and a retaining nib (182). Optionally, a line hook may further comprise a support member (188).

In a preferred embodiment, the upright member (186) has a height equal to at least the diameter of the IV line to be retained, the top member (184) attaches to said upright member (186) substantially perpendicularly and has a length at least the diameter of the IV line to be retained, and said retaining nib (182) substantially downwardly extends from said top member (184) substantially at the end of the top member (184) opposite its connection to said upright member (186). In this preferred embodiment, the retaining nib (182) may extend substantially downwardly from said to member (182) a distance such that the space between the bottom of said retaining nib (182) and said base plate (160) is smaller than the outer diameter of the IV line to be retained, such that said IV line must be forced into said line hook (180) by temporarily deforming one or more of said line hook (180) and said IV line and creating a friction fit between said line hook (180) and said IV line thereby. The number of line hooks (180) can be one, two, three, four, five, or more.

A line hook (180) is preferably made of material of strength and rigidity suitable to allow IV lines to he attached and removed multiple times without fatiguing or permanently deforming the structure of the line hook (180). Suitable materials include, by way of example, thermoform and thermoset plastics. The line hook (180) can be attached to said base plate (160) by any suitable mechanical means, as will be appreciated by one skilled in the art. In a preferred embodiment, the plurality of line hooks (180) are integrally molded or formed into said base plate (160).

Preferably, the outer surface of the ceiling of one or more line hooks (180) comprises a labeling surface (182) for labeling of the one or more IV lines retained or to he retained within a given line hook (180). Such labeling surface (182) can be labeled by any means known the art, including, without limitation, by writing, by marking, or by the placement of tags or stickers. In other embodiments, stickers, markings, or other identifying labels ma be placed on other portions of the line hooks (180) or on portions of the base plate (160) in con unction with one or more line hooks (180). In still other embodiments, all or portions of individual line hooks (180) or portions of the base plate (160) may be colored, with colors used to label individual line hooks.

The above specification, examples, and drawings provide a description of various embodiments of the apparatus claimed herein. As will be appreciated by one skilled in the art, various configurations, means of attachment, and materials can be used within the scope and spirit of the invention herein.

What is claimed is:

1. An apparatus for organizing IV lines, said apparatus comprising:
(a) a base-plate;
(b) one or more connectors attached to said base plate; and
(c) a plurality of line hooks attached to said base plate.

2. The apparatus of claim 1, wherein said one or more connectors comprise clamps adapted to mount to a bed rail.

3. The apparatus of claim 1, wherein said one or more connectors are adapted to mount to an IV pole.

4. The apparatus of claim 1, wherein said one or more connectors are adapted to mount to a wheelchair arm rail.

5. The apparatus of claim 2, wherein said one or more connectors are adapted to mount to a bed rail through friction fit.

6. The apparatus of claim 2, wherein said one or more connectors are integrally formed into said base plate.

7. The apparatus of claim 2, wherein said one or more connectors are attached to said base plate by a friction fit.

8. The apparatus of claim 2, wherein said apparatus comprises two connectors, and wherein said base plate comprises one connector substantially at each end.

9. The apparatus of claim 1, wherein each of said line hooks comprises an upright member, a top member attached and substantially perpendicular to said upright member, and a retaining nib substantially downwardly extending from said top member.

10. The apparatus of claim 9, wherein said upright member has a height equal to or greater than the outer diameter of be IV line to he retained.

11. The apparatus of claim 10, wherein said top member has a length equal to or greater than the outer diameter of the IV line to he retained.

12. The apparatus of claim 11, wherein said upright member has a height equal to or greater than twice the outer diameter of the IV line to be retained.

13. The apparatus of claim 11, wherein said top member has a length equal to or greater than twice the outer diameter of the IV line to be retained.

14. The apparatus of claim 11, wherein the height of said upright member is equal to or greater than the height defined by sum of the outer diameters of the IV lines to be retained in said line hook.

15. The apparatus of claim 11, wherein the length of said top member is equal to or greater than the height defined by sum of the outer diameters of the IV lines to be retained in said line hook.

16. The apparatus of claim 11, wherein one or more of said line hooks further comprises a labeling surface.

17. The apparatus of claim 11, wherein said apparatus comprises two or more line hooks.

18. The apparatus of claim 11, wherein one or more of said line hooks are adapted to retain one or more IV lines through a friction fit when the number of IV lines said line hook is adapted to retain are placed into said line hook.
